(19) United States
(12) Patent Application Publication
(10) Pub. No.: US 2013/0172208 A1
(43) Pub. Date: Jul. 4, 2013

(54) ASSESSMENT OF BONE MARROW RECOVERY BY MEASURING PLASMA EXOSOME mRNAS

(71) Applicants: HITACHI CHEMICAL COMPANY, LTD., Tokyo (JP); HITACHI CHEMICAL RESEARCH CENTER, INC., Irvine, CA (US)

(72) Inventor: Masato Mitsuhashi, Irvine, CA (US)

(73) Assignees: HITACHI CHEMICAL RESEARCH CENTER, INC., Irvine, CA (US); HITACHI CHEMICAL COMPANY, LTD., Tokyo (JP)

(21) Appl. No.: 13/733,805

(22) Filed: Jan. 3, 2013

Related U.S. Application Data
(60) Provisional application No. 61/583,064, filed on Jan. 4, 2012.

(51) Int. Cl. C12Q 1/68

(52) U.S. Cl. CPC C12Q 1/6876
USPC 506/9

(57) ABSTRACT

The present disclosure relates to the characterization of bone marrow conditions through the quantification of bone marrow-associated markers. In several embodiments, the bone marrow-associated markers are expressed in exosomes, which can be obtained from biological fluid samples, such as plasma or whole blood. In several embodiments, quantification of such markers allows for the assessment of bone marrow recovery following bone marrow transplantation.

### RELATED CASES

The contents of each priority document listed in the associated Application Data Sheet is incorporated in its entirety by reference herein.

### BACKGROUND

1. Field of the Invention

Several embodiments of the present disclosure relate to methods of characterizing a bone marrow condition through the quantification of markers from a blood sample from collected from an individual. Specifically, certain embodiments of the methods relate to the capture of exosomes, vesicles, and other circulating membrane bound nucleic acid and/or protein-containing structures that are released from the bone marrow into the bloodstream. After capture, markers that are specific to bone marrow cells are quantified in order to assess a bone marrow condition.

2. Description of the Related Art

Certain bone marrow (bone marrow) conditions are associated with dysfunctional production of cell populations found in the blood. For example, acute leukemia is a type of cancer affecting the body's blood-forming tissues, including the bone marrow. Acute leukemia is often characterized by a higher number of immature white blood cells. Complete blood count (CBC) can be used to diagnose leukemia, but a bone marrow aspiration is necessary to definitively rule out other conditions. Many other bone marrow conditions also require bone marrow aspiration for diagnosis, such as aplastic anemia (reduction in all blood cell types) and Hodgkin's lymphoma (cancer of lymph tissue).

These bone marrow conditions are often treated with bone marrow transplantation (bone marrow). bone marrow aspirations are often necessary to assess bone marrow recovery following bone marrow. bone marrow aspiration cannot be performed with high frequency due to the invasive and uncomfortable nature of the procedure, but there is a need to detect bone marrow recovery as soon as possible since patients are immunosuppressed prior to bone marrow.

### SUMMARY

In several embodiments, there is provided a method for identifying a subject exhibiting bone marrow recovery following hematopoietic precursor cell transplantation. In several embodiments, the transplantation may be of cord blood derived cells, bone marrow, and/or hematopoietic precursor cells derived from the bone marrow of a donor. In several embodiments, engineered precursor cells (e.g., induced pluripotent stem cells, iPS) are administered to a subject. iPS cells may be administered either in place of, or in addition to, other hematopoietic precursor cells. In several embodiments, the methods comprise obtaining one or more peripheral blood sample(s) comprising vesicles from the subject capturing at least a portion of the vesicles from the sample on or in a vesicle-capture material, thereby generating a vesicle sample, detecting expression of one or more mRNAs expressed by hematopoietic precursor cells in the bone marrow from the vesicle sample and identifying the subject as either not exhibiting bone marrow recovery when there is a lack of detection of expression of the one or more hematopoietic precursor cell-associated mRNAs or exhibiting bone marrow recovery when one or more of the hematopoietic precursor cell-associated mRNAs is detected. In several embodiments, the one or more blood samples are obtained within about 2 to about 4 weeks post-transplantation, a window in which conventional blood counts are inaccurate or unable to detect slight changes in bone marrow activity. In several embodiments, the samples are obtained within about 2 weeks post-transplantation.

In several embodiments, there is also provided a method for identifying a subject as responding to a therapy for a treatment of a blood disease resulting from dysfunctional bone marrow, comprising: obtaining at least a first and a second peripheral blood sample comprising vesicles from the subject, wherein the first sample is obtained before administration of a therapy to the subject for treatment of one or more blood diseases that result from dysfunctional bone marrow and wherein the second sample is obtained after the therapy is administered, capturing at least a portion of the vesicles from the samples on or in a vesicle-capture material, thereby generating at least two vesicle samples, detecting expression of one or more mRNAs expressed by hematopoietic precursor cells in the bone marrow from the vesicle sample and identifying the subject as either i) not responding to the therapy when there the expression of the one or more hematopoietic precursor cell-associated mRNAs is unchanged or decreased (in the second sample as compared to the first sample), or ii) responding to the therapy when expression one or more of the hematopoietic precursor cell-associated mRNAs is increased (in the second sample as compared to the first sample). In several embodiments, the blood diseases affecting the subject are those that result from dysfunctional bone marrow and/or insufficient quantities of functional bone marrow. Such diseases include, but are not limited to myelodysplastic syndrome, aplastic anemia, thrombocytopenia, leukopenia, granulocytopenia, pancytopenia, leukemia, and the like.

There are also provided herein methods for enabling a physician to recommend adjunct therapies to a subject based on the activity of the subject's bone marrow, comprising obtaining at least a first blood and a second sample from the subject, wherein the subject has received a hematopoietic precursor cell transplant, wherein the first blood sample is obtained prior to the transplant, wherein the blood samples comprise a plurality of vesicles comprising one or more hematopoietic precursor cell-associated mRNAs, quantifying expression of the one or more hematopoietic precursor cell-associated mRNAs in each of the first and second samples, comparing expression of the one or more hematopoietic precursor cell-associated mRNAs from the first sample with expression of the one or more mRNAs from the second sample, identifying the subject as either failing to exhibit bone marrow activity when there is a lack of detection of expression of the one or more hematopoietic precursor cell-associated mRNAs, or, exhibiting bone marrow activity when one or more of the hematopoietic precursor cell-associated mRNAs is detected; and indicating to the medical professional whether the subject is not exhibiting bone marrow activity or is exhibiting bone marrow activity, thereby enabling the medical professional to recommend adjunct therapies based on the activity of the bone marrow of the subject.

In several embodiments, there is provided a method for administering a one or more adjunct therapies to a subject who has received a hematopoietic precursor cell transplant, comprising obtaining at least a first blood and a second sample from said subject, wherein said first blood sample is obtained prior to said transplant and said second sample is obtained after said transplant, wherein said first and second blood samples comprise a plurality of vesicles comprising one or more hematopoietic precursor cell-associated mRNAs, requesting an analysis of the bone marrow activity of the subject, the test comprising quantifying expression of said one or more hematopoietic precursor cell-associated mRNAs in each of said first and second samples, comparing expression of said one or more hematopoietic precursor cell-associated mRNAs from said first sample with expression of said one or more mRNAs from said second sample, receiving the test results that identify said subject as i) not exhibiting bone marrow activity when there is a lack of detection of expression of said one or more hematopoietic precursor cell-associated mRNAs, or ii) exhibiting bone marrow activity when one or more of said hematopoietic precursor cell-associated mRNAs is detected; and if the subject is not exhibiting bone marrow activity, administering to said subject one or more adjunct therapies.

In several embodiments, the adjunct therapies comprise various other therapies administered to improve recovery from a transplant of hematopoietic precursor cells. In several embodiments, these adjunct therapies comprise anti-infective therapies to reduce the risk of infection after said transplant. In several embodiments, these adjunct therapies comprise administration of biological agents to stimulate blood cell production, such as, for example, erythropoietin for treating anemia, granulocyte colony stimulating factor for treating leukopenia, and thrombopoietin receptor agonist for treating thrombocytopenia, among others. Combinations of anti-infective and blood-cell stimulating therapies are also used, in several embodiments.

In several embodiments, an increase in the one or more hematopoietic precursor cell associated mRNAs in a second sample as compared to the first sample is correlated with an increase in bone marrow cell activity. In several embodiments, the increase in bone marrow cell activity is associated with bone marrow recovery after the hematopoietic precursor cell transplant.

In several embodiments a decrease in the one or more hematopoietic precursor cell associated mRNAs in the second sample as compared to the first sample is correlated with decrease in bone marrow cell activity. In several embodiments, the decrease in bone marrow cell activity is associated with loss of bone marrow function after the hematopoietic precursor cell transplant. In some embodiments, in which a bone marrow transplant is performed, the subject's loss of bone marrow function is due to rejection of donor bone marrow.

In several embodiments, the detection of expression of mRNAs is by one or more of a variety of methods that are able to detect nucleic acids, such as, for example, reverse-transcription polymerase chain reaction (RT-PCR), real-time RT-PCR, northern blotting, nucleic acid sequence-based amplification, invasive cleavage RNA assays, and/or branched DNA assays. Other methods of detecting RNA, DNA or proteins may also be used, including other gene amplification methods, multiple displacement amplification, fluorescence activated cell sorting, ELISA, mass spectrometry, and the like. In several embodiments, other methods may also be used. In some embodiments, non-mRNA-based methods are used in addition to, or in place of mRNA-based methods.

In several embodiments, the vesicles are selected from the group consisting of membrane particles, exosomes, exosome-like vesicles, and microvesicles. In several embodiments, the vesicles are selected from the group consisting of nanovesicles, vesicles, dexosomes, blebs, prostasomes, microparticles, intralumenal vesicles, endosomal-like vesicles and exocytosed vehicles. In one embodiment, the vesicles are exosomes.

In several embodiments, the blood sample comprises whole blood, while in some embodiments, the blood sample comprises plasma.

In several embodiments, the mRNAs are white blood cell-associated mRNAs. In some embodiments, the mRNAs are red blood cell-associated mRNAs. In several embodiments, the mRNAs are platelet-associated mRNAs. In some embodiments, the mRNAs are associated with more than one cell type. In several embodiments, the mRNAs are selected from the group consisting of B2M, ACTB, CD34, HBB, GATA1, UROD, THBS1, CD61, ITGA2B, PFKP, GPS, CD45, DEFA3, CD14, SRGN, CD3, CD8A, CD4, and CD19. In several embodiments, the mRNAs are selected from the group consisting of DEFA3, SRGN, CD61, ITGA2B, HBB, and UROD.

In several embodiments, the amount of the mRNAs from the subject is normalized to the amount of a control gene expressed by the subject. In other embodiments, the amount of the mRNAs from the subject are compared to amounts of corresponding mRNAs from a control population having known bone marrow status and/or known bone marrow function.

In several embodiments, there is provided a method for characterizing the condition (e.g., functional status) of a subject's bone marrow, comprising obtaining a peripheral blood sample comprising vesicles from the subject, capturing at least a portion of said vesicles from said blood sample, quantifying the expression levels of one or more RNAs associated with specific bone marrow cell types, wherein the expression level of the RNAs associated with specific bone marrow cell types is associated with the function and/or condition of the specific bone marrow cell types.

In several embodiments, such methods enable the characterization of a bone marrow condition as hyperplastic, hypoplastic, or normal. In several embodiments, the RNAs that are analyzed are specific to one or more of erythroblasts, myeloblast, megakaryocytes, or fibroblasts (among other cell types). As such, the methods allow the characterization of the bone marrow based on expression levels of those specific RNAs on exosomes that are collected peripherally. For example, increases in expression of red blood-cell specific markers results in the characterization of the bone marrow (in particular the bone marrow cells that are precursors to red blood cells) as hyperplastic. Similarly, a decrease in expression levels results in the characterization of the bone marrow as hypoplastic. Such characterization is important in some embodiments, for determining next therapeutic steps for a subject having had a bone marrow transplant.

In several embodiments there is provided a method of determining bone marrow recovery in a subject following bone marrow transplantation, comprising obtaining a peripheral blood sample comprising vesicles from the subject, capturing at least a portion of said vesicles from said sample on or in a vesicle-capture material, thereby generating a vesicle sample, detecting expression of one or more mRNAs expressed by hematopoietic precursor cells in the bone marrow from said vesicle sample, wherein detection of expression of said one or more hematopoietic precursor cell-associated mRNAs is associated with bone marrow recovery in said subject, and wherein a lack of detection of expression of said one or more hematopoietic precursor cell-associated mRNAs is associated with a lack of bone marrow recovery in said subject. Such methods are useful for transplant patients, as typically, the medical procedures that precede a transplant involve destruction of the subject's diseased or non-functional bone marrow (either in whole or in substantial part). As such, the subject will have little (e.g., indistinguishable from background levels) or no expression of hematopoietic precursor cell-associated mRNAs after the destruction of the endogenous bone marrow. Thus, the detection of any expression of a hematopoietic precursor cell-associated mRNA post-transplant indicates that the bone marrow is functional and recovering (e.g., non-rejected).

In several embodiments, there is also provided a method of assessing bone marrow recovery in a bone marrow transplant patient, comprising, obtaining a first and second blood sample from the subject, each sample comprising vesicles. In several embodiments, the second sample is collected at a time later than the first sample. This time may be several hours, though in other embodiments, the time is several days or weeks, and in some embodiments up to several months. The method also comprises capturing at least a portion of the vesicles from both blood samples; quantifying expression levels of one or more RNAs associated with specific bone marrow cell types; and comparing expression of said one or more bone marrow-associated RNAs from said first blood sample with expression of said one or more RNAs in vesicles from said second sample, wherein an increase in expression of said one or more bone marrow-associated RNAs in said second sample as compared to said first sample is associated with bone marrow recovery. In some embodiments, the samples are analyzed upon collection, while in other embodiments, the samples are stored frozen until an appropriate time for analysis. In still additional embodiments, additional samples are collected (e.g., serial samples are taken over time).

In several embodiments, the vesicles comprise one or more of membrane particles, exosomes, exosome-like vesicles, and/or microvesicles. In several embodiments, the vesicles comprise one or more of nanovesicles, vesicles, dexosomes, blebs, prostasomes, microparticles, intralumenal vesicles, endosomal-like vesicles and/or exocytosed vehicles. In one embodiment, the vesicles are exosomes.

In several embodiments the blood sample is whole blood. In some embodiments, plasma samples are prepared.

In several embodiments, the vesicles from said blood sample are captured on or in a vesicle-capture material. In several embodiments, the RNAs isolated and quantified are mRNAs.

In several embodiments, the RNAs are red blood cell-specific mRNAs. In some embodiments, the red blood cell-specific markers comprise one or more of HBB, GATA1, and UROD.

In several embodiments, the RNAs are platelet-specific mRNAs. In some embodiments, the platelet-specific mRNA comprises one or more of THBS1, CD61, ITGA2B, platelet-specific phosphofructokinase (PFKP), and GPS.

In several embodiments, the RNAs are white blood cell-associated mRNAs. In some embodiments, the RNAs are granulocyte-specific mRNAs. In some embodiments, the granulocyte-specific mRNAs comprise one or more of CD14, SRGN, and DEFA3.

In several embodiments, the amount of RNA from said subject is normalized to the amount of a control gene expressed by said subject in order to characterize the bone marrow function of the subject.

### BRIEF DESCRIPTION OF THE DRAWINGS

FIG. 1 depicts white blood cell (WBC) recovery in peripheral blood and myeloid cell-derived mRNAs (DEFA3 and SRGN) in plasma exosome. Day 0 is date of transplant. Arrow indicated early detection of WBC recovery by plasma exosome mRNA. Vertical dotted lines indicated the point where mRNA increased. Arrows indicate the early detection of white blood cell recovery via exosome analysis or by traditional cell count analysis.

FIG. 2 depicts platelet recovery in peripheral blood and megakaryocyte-derived mRNAs (ITGA2B and CD61) in plasma exosome. Vertical dotted lines indicated the point where mRNA increased. Arrows indicate the early detection of platelet recovery via exosome analysis or by traditional cell count analysis.

FIG. 3 depicts red blood cell (RBC) recovery in peripheral blood and erythroblast-derived mRNAs (HBB and UROD) in plasma exosome. Vertical dotted lines indicated the point where mRNA increased. Arrows indicate the early detection of red blood cell recovery via exosome analysis or by traditional cell count analysis.

FIGS. 4A-C depicts the slope of mRNA and complete blood count (CBC) recovery. Slope of mRNA increase (ΔCt/days, x-axis) was compared with that of CBC (y-axis). FIG. 4A depicts DEF3A (o) and SRGN (x) vs. WBC (×10³/μL). FIG. 4B depicts CD61 (o), ITGA2B (x) vs. platelet (×10⁴/μL). FIG. 4C depicts HBB (o), UROD (x) vs. reticulocyte

### DETAILED DESCRIPTION

While many diagnostic tests are performed on a biological fluid sample (e.g., blood, urine, etc.) extracted from a patient for the diagnosis or prognosis of disease, for bone marrow transplant patients, the most common procedure to evaluate the status of the bone marrow is collection of a bone marrow aspirate. This is because conclusions that might be drawn about bone marrow status based on complete blood counts (CBCs) are not only delayed, but may be inaccurate. For example, a CBC counts the currently circulating white blood cells (generally with differential counts for various white blood cell types), red blood cells, and platelets. However, these numbers are based on a snapshot of peripheral blood the moment of the blood draw, which cannot provide a direct conclusion as to the state of the hematopoietic precursor cells in the bone marrow. This is due, at least in part, to the inherent lag time that exists between a hematopoietic precursor cell differentiating into a mature cell, and the time at which that mature cell can be identified. For example, a person may present with a normal CBC at a first time, but at that same time, may also have a non-functional bone marrow, which will not be detected until blood counts begin to drop. Also confounding such an analysis is the different life-span of various blood cell types, which in some cases can further increase the lag time. Often, bone marrow aspirations can be interspersed with complete blood counts (CBCs), but CBCs alone are not sufficient to obviate the need for aspiration of bone marrow. Due to the highly invasive and painful nature of bone marrow aspirations, there is a need for less invasive, but accurate, diagnostics to assess the current condition of a patient's bone marrow.

Prior to transplanting donor bone marrow to a recipient, the recipient typically undergoes immunosuppression (e.g., chemotherapy, radiation, etc.) to prevent the recipient from rejecting the bone marrow transplanted from a donor and to destroy the subject's existing bone marrow, which is diseased, damaged, or otherwise non-functional. As a result, the subject will have no detectable markers related to hematopoietic precursor cells. After bone marrow transplantation, the donor's hematopoietic cells start to grow in the recipient's bone marrow cavity. A portion of the cells, when triggered by certain hormonal signals, will then begin to differentiate into one of multiple lineages to produce precursor cells for red blood cells (RBC) (erythroblast), white blood cell (WBC) (myeloblast, lymphoblast), and platelet (megakaryocyte), respectively. Again, based on certain hormonal signals, these immature “blast” cells, will eventually terminally differentiate into mature cells that are released into the peripheral blood. In the meantime, recipients are at a higher risk for developing life-threatening infections since they are immunosuppressed. The differentiation process may take days, or even weeks, before a recipient presents mature hematopoietic cells in their circulation. Thus, there exists a need to detect bone marrow status at an earlier time point. Thus, in several embodiments, among other applications of the methods disclosed herein, there is provided a method of determining bone marrow status in a subject following bone marrow transplantation, by obtaining a peripheral blood sample comprising vesicles from the subject, capturing at least a portion of the vesicles and expression of one or more mRNAs expressed by hematopoietic precursor cells in the bone marrow from the blood sample. In such cases, any detection of expression of markers associated with hematopoietic precursor cells is associated with bone marrow recovery in said subject. In contrast, a lack of detection of expression of one or more markers associated with hematopoietic precursor cells is associated with a lack of bone marrow recovery in said subject.

While detection of hematopoietic precursor cells would be an ideal method to assess bone marrow recovery/status, CBC cannot be used in this manner, as the hematopoietic precursor cells do not migrate into peripheral blood, but remain in the bone marrow until they are more mature. Thus, in order to quantify hematopoietic precursor cells within the bone marrow, the clinician must perform a bone marrow aspiration, which is a painful, invasive procedure. During a bone marrow aspiration, a clinician inserts a hollow needle through the bone and into the bone marrow. The clinician then withdraws a sample of bone marrow using a syringe attached to the needle. Several samples may need to be taken at one time, though often, multiple procedures are needed over time. Thus, there exists a need to develop a less invasive procedure to characterize a bone marrow status.

Several embodiments of the present invention provide an effective test for assessing bone marrow status, in particular status after transplantation, without requiring bone marrow aspiration. In several such embodiments, bone marrow recovery after transplantation is assessed by the analysis of exosomes released from hematopoietic precursor cells in the bone marrow. As discussed above, the hematopoietic precursor cells exist in bone marrow, however exosomes shed from those cells migrate into peripheral blood. Thus, the methods disclosed herein allow for assessment of a bone marrow condition by quantifying specific markers of bone marrow cells (e.g., hematopoietic precursor cells in the bone marrow) in exosomes captured from peripheral blood.

In several embodiments, the methods disclosed herein enable a medical provider to assess the bone marrow recovery status of an individual after a bone marrow transplant. In several embodiments, the methods can be performed before a transplant, in order to evaluate the type and or relative aggression of therapy needed to ablate the existing bone marrow. In several embodiments, the methods disclosed herein allow a medical provider to recommend certain adjunct therapies to a bone marrow transplant recipient. For example, post-transplant immune boosting therapies may be recommended if bone marrow recovery or activity is less than desired. Therapies may include pharmacologic, holistic or other approaches. In several embodiments, the methods provided herein allow ex vivo and substantially less invasive determination of bone marrow function as compared to aspiration-based methods. Such methods may optionally still be used in conjunction with those disclosed herein In several embodiments, the methods allow a prediction to be made on whether a subject is likely to reject transplanted bone marrow (or is presently rejecting transplanted bone marrow).

Identification of specific biomarkers including, but not limited to, DNA, RNA (such as mRNA, miRNA or microRNA, and siRNA), and proteins can provide bio-signatures that are used for the assessment of bone marrow condition (or for the diagnosis, prognosis, or theranosis of another condition or disease). While DNA and RNA typically are contained in the intracellular environment, these nucleic acids also exist extracellularly. In some cases, DNA and/or RNA are naked (e.g., not encapsulated or associated with another structure or compound. RNAses, which degrade RNA, are known to be elevated in some disease states, for example, in certain cancers. The extracellular environment, including the plasma, serum, urine, or other biological fluids is known to contain substantial quantities of RNAses. Given this context, extracellular DNA, RNA, or other biomarkers are often considered a meaningless degradation product in an extracellular sample, not only because their levels may not be representative of the true levels of the intracellular message, but also due to the instability and poor quality of the nucleic acids.

Moreover, due to the rapid rate of nucleic acid degradation in the extracellular environment, conventional understanding suggests that many tissues are unable to provide nucleic acid that would be suitable as a diagnostic target, because the nucleic acids would be degraded before they could be used as a template for detection. However, extracellular RNA (as well as other biomarkers disclosed herein) is often associated with one or more different types of vesicles, such as membrane particles (ranging in size from 50-80 nm), exosomes (ranging in size from 50-100 nm), exosome-like vesicles (ranging in size from 20-50 nm), and microvesicles (ranging in size from 100-1000nm). Other vesicle types may also be captured, including, but not limited to nanovesicles, vesicles, dexosomes, blebs, prostasomes, microparticles, intralumenal vesicles, endosomal-like vesicles or exocytosed vehicles. As used herein, the terms “exosomes” and “vesicles” shall be given their ordinary meaning and shall also be read to include any shed membrane bound particle that is derived from either the plasma membrane or an internal membrane. For clarity, the terms describing various types of vesicles shall, unless expressly stated otherwise, be generally referred to as vesicles or exosomes. Exosomes can also include cell-derived structures bounded by a lipid bilayer membrane arising from both herniated evagination (blebbing) separation and sealing of portions of the plasma membrane or from the export of any intracellular membrane-bounded vesicular structure containing various membrane-associated proteins of tumor origin, including surface-bound molecules derived from the host circulation that bind selectively to the tumor-derived proteins together with molecules contained in the exosome lumen, including but not limited to tumor-derived microRNAs or intracellular proteins. Exosomes can also include membrane fragments. Circulating tumor-derived exosomes (CTEs) as referenced herein are exosomes that are shed into circulation or bodily fluids from tumor cells. CTEs, as with cell-of-origin specific exosomes, typically have unique biomarkers that permit their isolation from bodily fluids in a highly specific manner. As achieved by several embodiments disclosed herein, selective isolation of any of such type of vesicles allows for isolation and analysis (e.g., quantification) of their RNA (e.g., mRNA, microRNA, and siRNA, etc.), or other nucleic acids/protein, which can be useful in assessing the condition of the bone marrow by measuring one or more markers of hematopoietic precursor cells (as well as diagnosis or prognosis of numerous other diseases).

Conventional methods for isolation of exosomes, or other vesicles, often involve ultracentrifugation (often multiple rounds) in order to separate the vesicles from other matter in a biological sample. There exist devices, compositions, and methods for capture of exosomes, vesicles, and other circulating membrane bound, nucleic acid (including, but not limited to DNA, RNA, mRNA, microRNA, and siRNA) and/or protein-containing structures that are released from cells into biological fluids which are advantageous in several of the methods disclosed herein. Additional information about such devices, compositions, and methods can be found in International Patent Application No: PCT/US2011/040076, filed on Jun. 10, 2011, which is incorporated in its entirety by reference herein.

In several embodiments, isolation of exosomes that comprise specific biomarkers that are to be analyzed is advantageous because access to such biomarkers is typically limited by access to the source tissue (for example, the hematopoietic precursor cells that are within the bone marrow). In other words, certain tissues (e.g., internal organs, bone marrow, etc) from which an invasive biopsy (or other surgical approach) is typically obtained in order to analyze biomarker expression can benefit from the methods disclosed herein, which allow for biomarker analysis with a less invasive procedure. The disclosed methods can then be used to characterize (e.g., diagnose) particular conditions from more easily (and less painfully) obtained samples, than would otherwise be possible with traditional methods.

As a result of the quantification of cell-specific markers according to the methods disclosed herein, a variety of bone marrow conditions can be identified in a subject. For example, several embodiments are useful in diagnosis of acute leukemia by quantifying one or more markers for hematopoietic precursor cells within the bone marrow to determine whether there are abnormal numbers of immature WBCs. The method can also be used to diagnose aplastic anemia by quantifying one or more markers for bone marrow cells to determine whether there is an abnormally low number of bone marrow precursor cells. The methods described herein are also used, in several embodiments, to assess bone marrow condition following treatment of both of these conditions, as well as other bone marrow conditions.

Several embodiments provide a method of characterizing the bone marrow status in a subject (e.g., determining if production of a certain lineage of blood cell is hyperplastic, hypoplastic, or normal) based on analysis of blood-cell specific markers on exosomes released from precursor cells that dwell within the bone marrow cavity. As discussed herein, advantageously, these methods are not dependent on obtaining a bone marrow aspirate sample (though in some embodiments, a bone marrow sample may optionally be obtained to further assess the condition of the marrow). In several embodiments, one or more peripheral blood sample (whole blood in some embodiments and plasma in other embodiments) is collected and vesicles are isolated from the blood sample. RNA (e.g., mRNA, though other types of RNA can be analyzed) is isolated from the vesicles. In several embodiments isolated RNA are specific to particular types of hematopoietic cells within the bone marrow. RNAs that are isolated and analyzed may be specific to, for example, erythroblasts, myeloblast, megakaryocytes, or fibroblasts (among other cell types) which enables the characterization of the bone marrow based on identification and expression levels of those specific RNAs. For example, HBB, GATA1, and UROD are expressed specifically on red blood cells, among other markers. THBS1, CD61, ITGA2B, PFKP, and GP5 are expressed specifically on platelets, among other markers. CD45, DEFA3, CD14, SRGN, CD3, CD8A, CD4, and CD19 are expressed specifically on WBCs, among other markers. Thus, the methods disclosed herein are used to quantify the expression of one or more of these specific markers, which, if present, indicate that the bone marrow is being stimulated and is actively producing precursor cells from those specific lineages. Likewise, lack of expression of these markers indicates a lack of activity in bone marrow cells with respect to generation of that particular lineage of hematopoietic cell.

In several embodiments, additional peripheral blood samples are taken serially over the course of time to capture vesicles and thus to monitor the status of a subject's bone marrow, particularly after bone marrow transplant. In certain embodiments, this time is several hours, though in other embodiments, the time is several days or weeks, and in some embodiments up to several months. RNA (e.g., mRNA, though other types of RNA and/or other biomarkers are analyzed in other embodiments) is isolated from the vesicles. In several embodiments the isolated RNA is specific to particular types of hematopoietic precursor cells. RNAs that are isolated and analyzed may be specific to erythroblasts, myeloblast, megakaryocytes, or fibroblasts (among other cell types) which enables the characterization of the current status of the subject's bone marrow based on identification and expression levels of, for example, the precursor cell-type specific RNAs as discussed above. Comparison of the expression of the markers in the serial samples is then used to determine the status of the bone marrow over time. Such embodiments are advantageous, for example, to monitor the status of a bone marrow transplant patient during a period of immunosuppression, in order to determine if particular measures are warranted to maintain the subject in an infection-free state. In several embodiments, changes in the expression of one or more markers are associated with improved (or diminished) bone marrow activity. In some embodiments, statistically significant changes are detected across samples over time. The statistical significance of the difference in amount of expression can be determined in any of a number of ways that are well-known to those having ordinary skill in the art. As but one example of an appropriate test for statistical significance, statistical p values can be calculated by t-test and identified as significant when p≦0.05.

In several embodiments, the exosomes (or other biomarker-associated membrane bound vesicles) are isolated, enriched, or otherwise concentrated, from the blood sample in order to increase the yield of exosomes. In several embodiments, increased yield is achieved simply by applying multiple sample aliquots to an exosome capture device (such as those disclosed in PCT/US2011/040076, filed on Jun. 10, 2011, which is incorporated by reference in its entirety herein.

In several embodiments, blood samples are passed through devices comprising a sample loading region, one or more vesicle-capturing materials, and a sample receiving region. The devices allow the blood sample to be loaded into the sample loading region, passed over or through the vesicle-capturing material in order to trap, temporarily hold, or otherwise isolate the vesicles from the remainder of the components of the blood sample, which is received in the sample receiving region of the device. In some embodiments, the device comprises a single sample loading region, one or more vesicle-capturing materials, and a single sample receiving region. In several such embodiments, the devices are provided in a single use format (e.g., are pre-sterilized and disposable). However, in some embodiments, after capture of the vesicles and subsequent processing, the device can be cleaned and/or sterilized, and re-used. In several embodiments, the device comprises a plurality of sample loading regions, each associated with a corresponding plurality of vesicle-capturing material(s), and a corresponding plurality of sample receiving regions. In some embodiments, multi-well devices are configured with standard dimensions (e.g., those of a 96 well or 384 well plate) such that the device can be placed in standard laboratory equipment (e.g., a standard low-speed plate centrifuge). In still additional embodiments, the device is configured to interact with a device for high-throughput quantification of mRNA such as those described in U.S. Pat. No. 7,745,180, issued on Jun. 29, 2010, and is incorporated be reference herein.

In some embodiments, the vesicle capture material is modified in order in enhance its vesicle capturing capability or to enable capture of different types of vesicles (e.g., electrocharged, chemically modified, or biologically modified capture materials may be used, in several embodiments. In some embodiments, the capture material is configured to recognize vesicle markers comprising non-proteins such as lipids, carbohydrates, nucleic acids, RNA, mRNA, siRNA, microRNA, DNA, etc., in particular those associated with hematopoietic precursor cells located in the bone marrow.

Depending on the configuration of the vesicle capturing device, various procedures may be used to pass a biological sample through the capture material. For example, in one embodiment, low speed centrifugation is used. In one embodiment, vacuum pressure is used. In one embodiment, positive pressure is used. In one embodiment, gravity is used. Combinations of these procedures may also be used.

In several embodiments, a peripheral blood sample is obtained from a patient in order to characterize the function of the patient's bone marrow. In several embodiments, the blood is whole blood. In several embodiments, the blood sample is heparinized (either during, or after collection). The blood sample can be used with pretreatment or can be used “as is”, e.g., without pretreatment. When pretreatment is used, it can take many forms, including sample fractionation, precipitation of unwanted material, etc. For example, some embodiments allow for samples to be taken from donors and used “as-is” for isolation and testing of biomarkers. However, some embodiments allow a user to pretreat samples for certain reasons. These reasons include, but are not limited to, protocols to facilitate storage, facilitating biomarker detection, etc.

A variety of different biomarkers are analyzed that can be used to characterize the bone marrow condition of a subject. In several embodiments, the biomarkers are analyzed by quantification of gene expression. In some embodiments, polymerase chain reaction, including reverse-transcription polymerase chain reaction (RT-PCR) is used. In several embodiments, real-time reverse transcriptase polymerase chain reaction is used. In other embodiments, northern blot analysis, fluorescence activated cell sorting, ELISA, mass spectrometry, or combinations thereof are used. In some embodiments, protein biomarkers are analyzed by protein array analysis, western blotting, or other protein-directed analysis method. Other quantification methods may also optionally be used. In several embodiments, the quantifying comprises use of real-time RT-PCR.

In several embodiments, myelocyte-related genes (for example, serglycin (SRGN), defensin A3 (DEFA3)). Other hemoglobin genes (HBB), erythroblast-specific genes (for example, globin transcription factor 1 (GATA1), uroporphyrinogen decarboxylase (UROD)), megakaryocyte-specific genes (for example, thrombospondin 1 (THBS1), platelet glycoprotein IIb of IIb/IIIa complex (ITGA2B), platelet-specific phosphofructokinase (PFKP), platelet glycoprotein V (GP5)), CD61, or combinations thereof are evaluated in several embodiments.

In some embodiments, an abnormal bone marrow condition itself induces differences in the amount of exosomes produced in an individual. However, exosome production variance may not be tissue-specific. As such, in several embodiments, the analysis of markers of premature hematopoietic cells involves normalization of the bone marrow-associated biomarker by the expression level of an appropriate control gene. In some embodiments, however, normalization is not performed. In still additional embodiments, normalization is made relative to normal samples (e.g., a plurality of samples from non-diseased individuals).

### EXAMPLE

Examples provided below are intended to be non-limiting embodiments of the invention.

### Example 1

### Assessment of Bone Marrow Status by the Quantification of Bone Marrow-Derived Biomarkers from Bone Marrow Transplant Subjects

The present example employed mRNA quantification of bone marrow-derived biomarkers associated with bone marrow recovery in 18 cases of bone marrow transplant. Exosome-capture membranes, such as those disclosed in PCT/US2011/040076, filed on Jul. 10, 2011, and incorporated in its entirety by reference herein, were used to capture exosomes before and after bone marrow transplant and assembled to 96-well filterplates to make the assay system high throughput. Plasma samples were collected from bone marrow transplant patients and applied to the filterplate. Subsequently, trapped exosomes were lysed on the membrane, and poly(A)+ RNA was purified by transferring lysates to the oligo(dT)-immobilized 96-well plate, followed by cDNA synthesis and PCR (as discussed in U.S. Pat. No. 7,745,180, issued on Jun. 29, 2010, and is incorporated be reference herein). The results of real time PCR were expressed as the cycle threshold (Ct) (lower number equivalent to higher expression) and compared with CBC. The target mRNAs are listed in Table 1, below.

TABLE I
List of target mRNAs.

| Category | Gene | Description |
|---|---|---|
| Control | B2M | β2 microglobulin |
| | ACTB | β actin |
| Stern cell | CD34 | CD34 |
| RBC | HBB | β hemoglobin |
| | GATA1 | globin transcription factor 1 |
| | UROD | uroporphyrinogen decarboxylase |
| Platelet | THBS1 | thrombospondin 1 |
| | CD61 | β3 integrin (platelet glycoprotein IIIa) |
| | ITGA2B | α2b integrin (platelet glycoprotein IIb) |
| | PFKP | platelet phosphofructokinase, |
| | GP5 | platelet glycoprotein V |
| pan-WBC | CD45 | Protein tyrosine phosphatase, receptor type, C |
| Granulocyte | DEFA3 | α3 defensin, neutrophil-specific |
| | CD14 | CD14 |
| | SRGN | Serglycin (secretory granule proteoglycan core |
| | | peptide) |
| T-cell | CD3 | CD3 |
| | CD8A | CD8A |
| | CD4 | CD4 |
| B-cell | CD19 | CD19 |

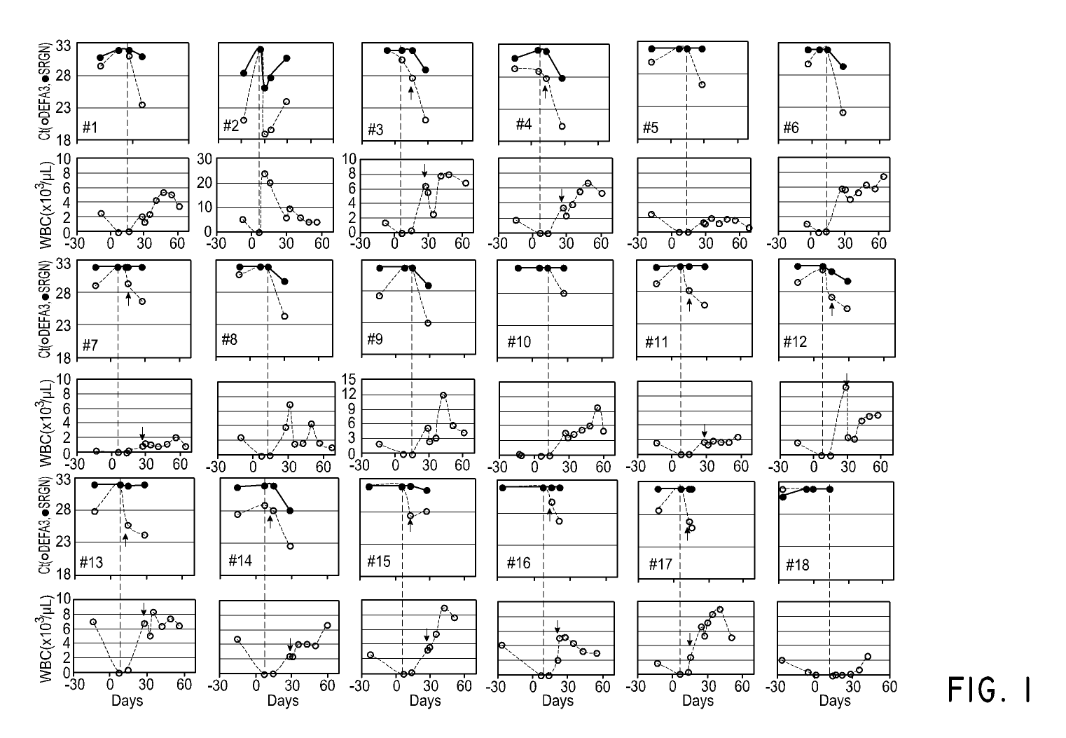

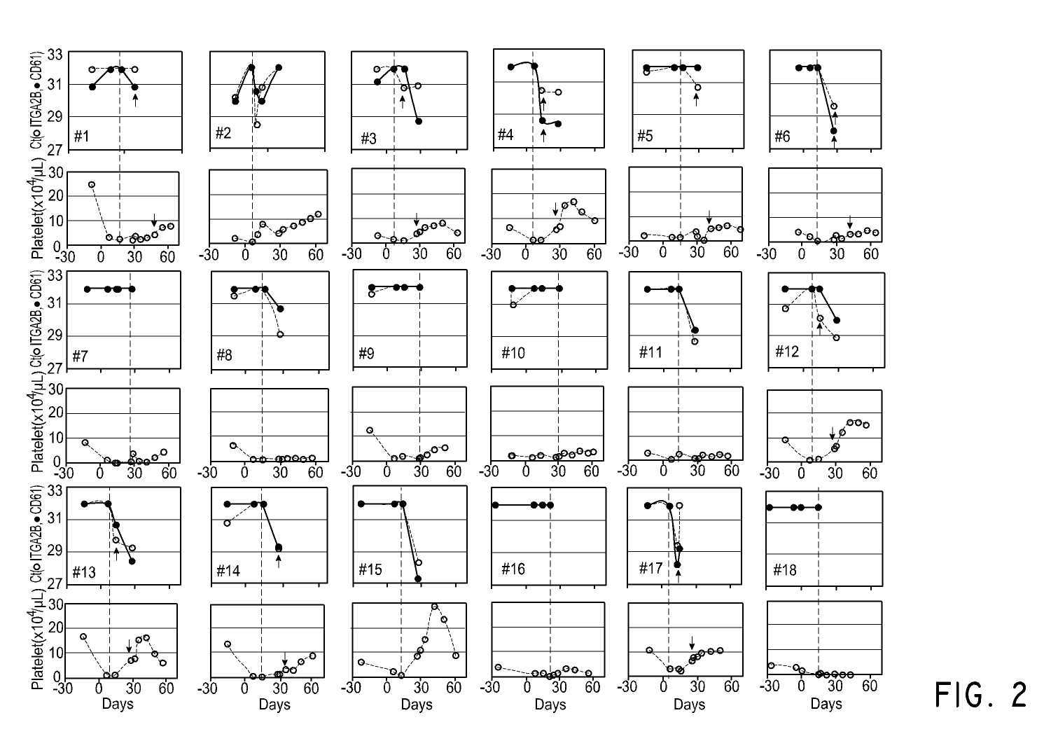

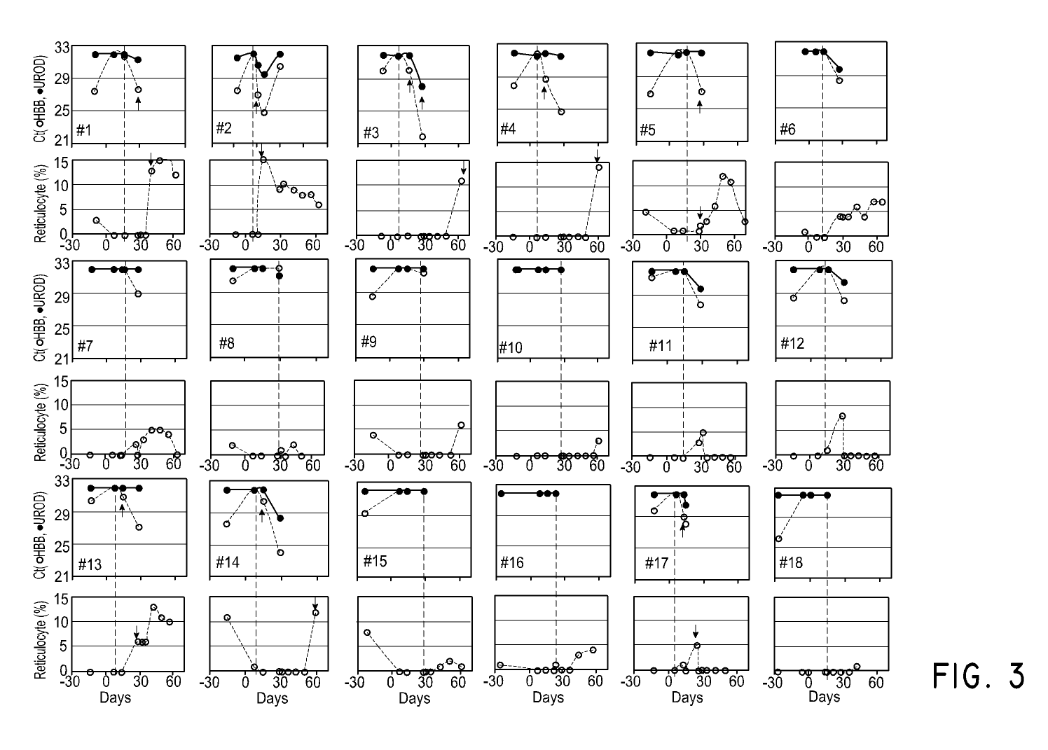

Expression of various markers captured from exosomes are shown in FIGS. 1-3. FIG. 1 depicts results of DEFA3 (∘) and SRGN () mRNA expression correlated to white blood cell count. FIG. 1 depicts time-correlated expression of these genes versus WBC counts (similar layouts are used for FIGS. 2 and 3). FIG. 2 depicts ITGA2B (∘) and CD61 () mRNA expression correlated with platelet counts. FIG. 3 depicts the expression of red blood cell specific markers HBB (∘) and UROD () correlated to reticulocyte counts (%). As shown in these figures, the sensitivity of the markers tested was variable. For example, the detection of DEFA3 was greater than SRGN, and more robust in predictive value (e.g., DEFA3 expression increases were detectable prior to increased white blood cell counts). Likewise, increases in HBB expression were more readily detected as compared to UROD, for reticulocytes. For platelets, CD61 expression was similar to that of ITGA2B. Thus, in several embodiments, these genes with higher levels of expression (e.g., DEFA3 and/or HBB) are preferred in diagnostics to assess bone marrow condition. However in other embodiments, one or more of the mRNAs tested in this example that were deemed to be less sensitive are used in diagnostic assays.

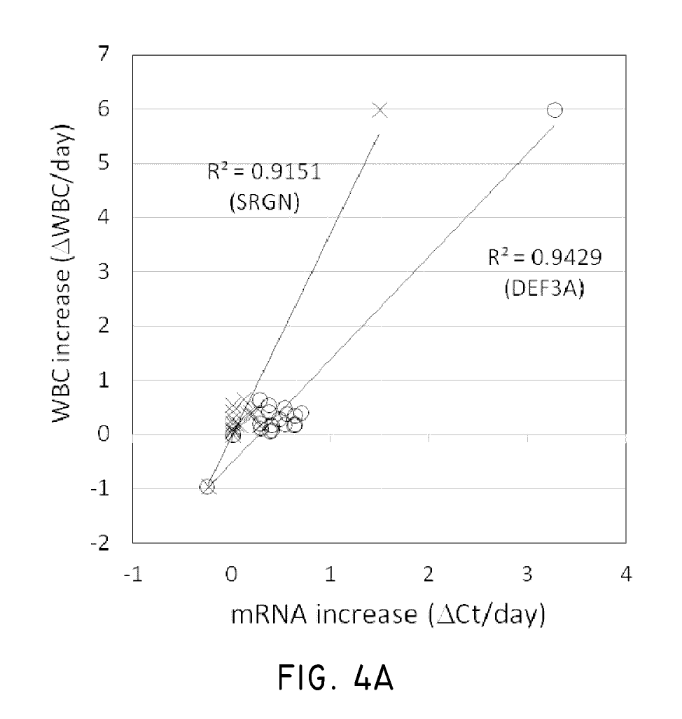

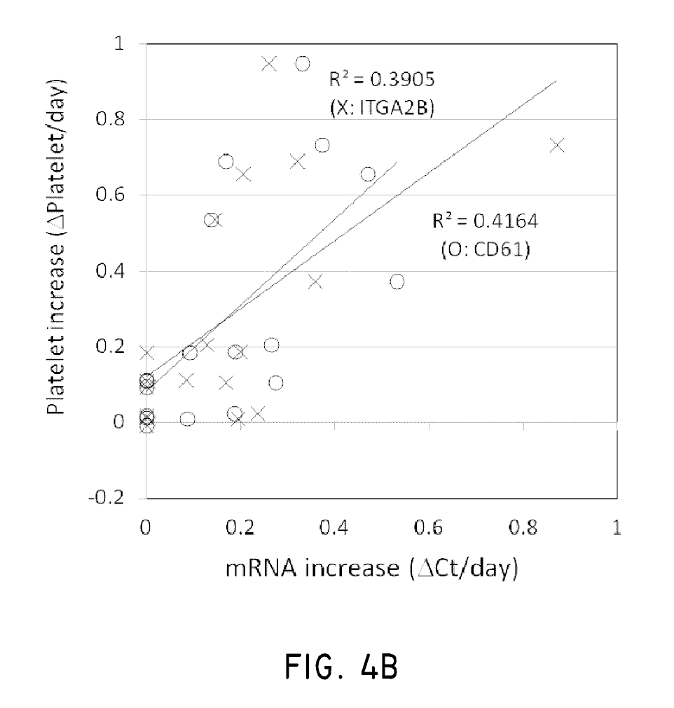

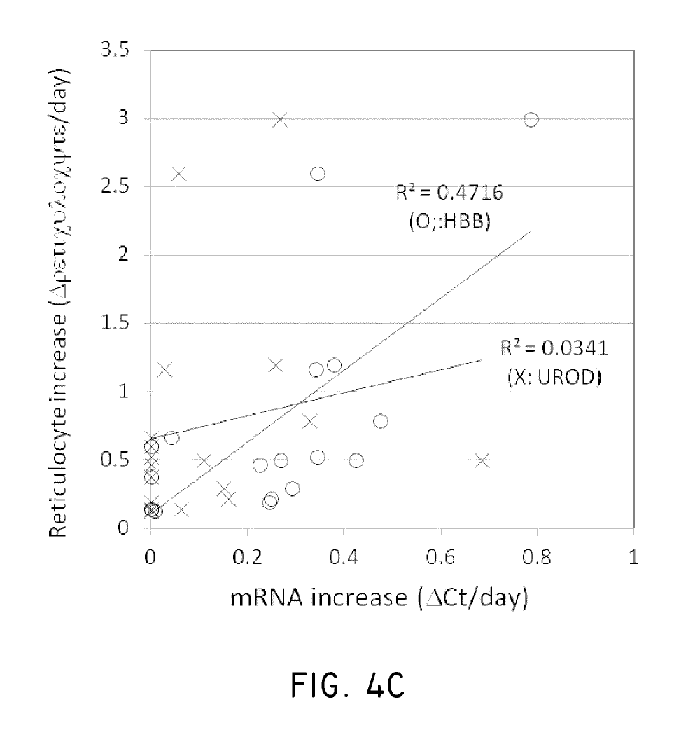

Of the 17 cases that showed increased white blood cell mRNA, all 17 cases demonstrated an increase in white blood cells, and the one case that did not show an increase in mRNA also failed to show an increase in white blood cells (Table II). Out of the 17 cases that showed an increase in both mRNA and white blood cells, seven cases showed simultaneous increase in both mRNA and white blood cells, whereas mRNA increase was earlier than that of white blood cells in the remaining ten cases with a lag time of 6.7±1.8 days (Table II). The slope of mRNA increase (DCt/day) was well correlated with the slope of WBC (DWBC/day) with r² more than 0.9 for both DEFA3 and SRGN (FIG. 4A). These data therefore indicate that detection of white blood cell associated mRNA from exosomes obtained from peripheral blood samples is useful in assessing the condition of that subject bone marrow.

Similarly, mRNA results correlated with that of platelet counts, with detectable increases in mRNA levels happening earlier than increases in platelet counts detected by CBC (see e.g., Table II). Eleven cases showing increased platelet-related mRNA also demonstrated an increase in platelet count. Three cases that did not show an increase in platelet-related mRNA also failed to show an increase in platelet count. Out of the eleven cases that showed an increase in both mRNA and platelet, two cases showed simultaneous increase in both mRNA and platelet, whereas mRNA increase was earlier than that of platelet in the remaining nine cases with a lag time of 12.3±7.3 days (Table II). The slope of mRNA increase (DCt/day) of CD61 and ITGA2B correlated with the slope of platelet (D/day) with r² more than 0.39 (FIG. 4B). Again, these results show that detection of mRNA from exosomes obtained from peripheral blood can be used to assess the status of the patient's bone marrow prior to the time at which a traditional blood count could be used to assess bone marrow status.

With respect to reticulocytes, 12 cases showing increased reticulocyte-related mRNA also demonstrated an increase in reticulocyte count. Two cases that did not show an increase in reticulocyte-related mRNA also failed to show an increase in reticulocyte count. Out of the 12 cases that showed an increase in both mRNA and reticulocyte, four cases showed simultaneous increase in both mRNA and reticulocyte, whereas mRNA increase was earlier than that of reticulocyte in the remaining eight cases with a lag time of 22.4±16.9 days (Table II). The slope of mRNA increase (DCt/day) of HBB correlated with the slope of reticulocyte (D/day) with r² more than 0.47 (FIG. 4B). These results further demonstrate that the methods disclosed herein are effective at accurately assessing the status of bone marrow by identification increased mRNA levels that are associated with specific hematopoietic precursor cells that resident in the bone marrow.

TABLE II
Summary of Results

| | | mRNA | | Lag time | | Days |
|---|---|---|---|---|---|---|
| | | → | ↑ | mRNA = WBC | mRNA→WBC | mean ± s.d. |
| WBC | → | 1 | 0 | | | |
| | ↑ | 0 | 17 | 7 | 10 | 6.7 ± 1.8 |
| Platelet | → | 3 | 2 | | | |
| | ↑ | 2 | 11 | 2 | 9 | 12.3 ± 7.3 |
| Reticulo- | → | 2 | 0 | | | |
| cyte | ↑ | 4 | 12 | 4 | 8 | 22.4 ± 16.9 |

The above example shows successful quantification of bone marrow cells-derived poly(A)+ RNA in plasma exosome from peripheral blood. Thus, in several embodiments, the status of bone marrow recovery is characterized by an increase or decrease in expression of one or more markers for bone marrow cells. In some embodiments, detection of successful bone marrow is identified if expression of one or more markers for bone marrow cells in a sample is elevated as compared to an earlier collected sample. While bone marrow aspiration may be the historical diagnosis of choice, the methods disclosed herein, in several embodiments have the potential to accurately and less invasively assess a bone marrow condition, as well as reduce the number of bone marrow aspirations during treatment follow up.

Various modifications and applications of embodiments of the invention may be performed, without departing from the true spirit or scope of the invention. Further, the disclosure herein of any particular feature, aspect, method, property, characteristic, quality, attribute, element, or the like in connection with an embodiment can be used in all other embodiments set forth herein. Method steps disclosed herein need not be performed in the order set forth. It should be understood that the invention is not limited to the embodiments set forth herein for purposes of exemplification, but is to be defined only by a reading of the appended claims, including the full range of equivalency to which each element thereof is entitled.

What is claimed is:

1. A method for identifying a subject exhibiting bone marrow recovery following hematopoietic precursor cell transplantation, comprising:
(a) obtaining at least one peripheral blood sample comprising vesicles from the subject,
(b) capturing at least a portion of said vesicles from said sample on or in a vesicle-capture material, thereby generating a vesicle sample;
(c) detecting expression of one or more mRNAs expressed by hematopoietic precursor cells in the bone marrow from said vesicle sample by a method selected from the group consisting of reverse-transcription polymerase chain reaction (RT-PCR), real-time RT-PCR, northern blotting, nucleic acid sequence-based amplification, invasive cleavage RNA assay, and branched DNA assay; and
d) identifying said subject as:
i) not exhibiting bone marrow recovery when there is a lack of detection of expression of said one or more hematopoietic precursor cell-associated mRNAs, or
ii) exhibiting bone marrow recovery when one or more of said hematopoietic precursor cell-associated mRNAs is detected.

2. The method of claim 1, wherein the vesicles are selected from the group consisting of membrane particles, exosomes, exosome-like vesicles, microvesicles, nanovesicles, vesicles, dexosomes, blebs, prostasomes, microparticles, intralumenal vesicles, endosomal-like vesicles and exocytosed vehicles.

3. The method of claim 1, wherein the vesicles are exosomes.

4. The method of claim 1, wherein said blood sample comprises whole blood.

5. The method of claim 1, wherein said blood sample comprises plasma.

6. The method of claim 1, wherein said mRNAs are white blood cell-associated mRNAs.

7. The method of claim 1, wherein said mRNAs are red blood cell-associated mRNAs.

8. The method of claim 1, wherein said mRNAs are platelet-associated mRNAs.

9. The method of claim 1, wherein said mRNAs are selected from the group consisting of B2M, ACTB, CD34, HBB, GATA1, UROD, THBS1, CD61, ITGA2B, PFKP, GP5, CD45, DEFA3, CD14, SRGN, CD3, CD8A, CD4, and CD19.

10. The method of claim 10, wherein said mRNAs are selected from the group consisting of DEFA3, SRGN, CD61, ITGA2B, HBB, and UROD.

11. The method of claim 1, wherein said amount of said mRNAs from said subject is normalized to the amount of a control gene expressed by said subject.

12. The method of claim 1, wherein said at least one peripheral blood sample is obtained within about two weeks of said hematopoietic precursor cell transplantation.

13. A method for identifying a subject as responding to a therapy for a treatment of a blood disease resulting from dysfunctional bone marrow, comprising:
(a) obtaining at least a first and a second peripheral blood sample comprising vesicles from the subject,
wherein said first blood sample is obtained prior to administration to said subject of a therapy for one or more blood diseases that result from dysfunctional bone marrow,
wherein said second blood sample is obtained after administration of said therapy;
(b) capturing at least a portion of said vesicles from said samples on or in a vesicle-capture material, thereby generating at least two vesicle samples;
(c) detecting expression of one or more mRNAs expressed by hematopoietic precursor cells in the bone marrow from said vesicle samples by a method selected from the group consisting of reverse-transcription polymerase chain reaction (RT-PCR), real-time RT-PCR, northern blotting, nucleic acid sequence-based amplification, invasive cleavage RNA assay, and branched DNA assay; and
d) identifying said subject as:
i) not responding to said therapy when the expression of said one or more hematopoietic precursor cell-associated mRNAs is unchanged or decreased in said second blood sample as compared to said first blood sample, or
ii) responding to said therapy when the expression of one or more of said hematopoietic precursor cell-associated mRNAs is increased in said second blood sample as compared to said first blood sample.

14. The method of claim 13, wherein said one or more blood diseases that result from dysfunctional bone marrow are selected from the group consisting of myelodysplastic syndrome, aplastic anemia, thrombocytopenia, leukopenia, leukemia.

15. A method for enabling a medical provider to recommend adjunct therapies to a subject who has received a hematopoietic precursor cell transplant, comprising:
obtaining at least a first blood and a second sample from said subject,
wherein said first blood sample is obtained prior to said transplant and said second sample is obtained after said transplant,
wherein said first and second blood samples comprise a plurality of vesicles comprising one or more hematopoietic precursor cell-associated mRNAs;
quantifying expression of said one or more hematopoietic precursor cell-associated mRNAs in each of said first and second samples by a method selected from the group consisting of reverse-transcription polymerase chain reaction (RT-PCR), real-time RT-PCR, northern blotting, nucleic acid sequence-based amplification, invasive cleavage RNA assay, and branched DNA assay;
comparing expression of said one or more hematopoietic precursor cell-associated mRNAs from said first sample with expression of said one or more mRNAs from said second sample,
identifying said subject as i) not exhibiting bone marrow activity when there is a lack of detection of expression of said one or more hematopoietic precursor cell-associated mRNAs, or ii) exhibiting bone marrow activity when one or more of said hematopoietic precursor cell-associated mRNAs is detected; and
indicating to said medical professional whether the subject is not exhibiting bone marrow activity or is exhibiting bone marrow activity, so as to enable the medical professional to recommend an adjunct therapy if the subject is not exhibiting bone marrow activity.

16. The method of claim 15, wherein said adjunct therapies comprise anti-infective therapies to reduce the risk of infection after said transplant, biological agent therapies to stimulate blood cell production, and combinations thereof.

17. The method of claim 15, wherein an increase in said one or more hematopoietic precursor cell associated mRNAs in said second sample as compared to said first sample is correlated with an increase in bone marrow cell activity.

18. The method of claim 17, wherein said increase in bone marrow cell activity is associated with bone marrow recovery after said hematopoietic precursor cell transplant.

19. The method of claim 15, wherein a decrease in said one or more hematopoietic precursor cell associated mRNAs in said second sample as compared to said first sample is correlated with decrease in bone marrow cell activity.

20. The method of claim 19, wherein said decrease in bone marrow cell activity is associated with loss of bone marrow function after said hematopoietic precursor cell transplant.

21. The method of claim 20, wherein said loss of bone marrow function is due to rejection of donor bone marrow.
